(19) United States
(12) Patent Application Publication
(10) Pub. No.: US 2011/0064806 A1
(43) Pub. Date: Mar. 17, 2011

(54) SOLID COMPOSITIONS COMPRISING AN OXADIAZOANTHRACENE COMPOUND AND METHODS OF MAKING AND USING THE SAME

(75) Inventors: Dharma Rao Polisetti, High Point, NC (US); Eric Benjamin, Jamestown, NC (US); James C. Quada, JR., High Point, NC (US); Thorsteinn Thorsteinsson, Jamestown, NC (US)

(73) Assignee: TransTech Pharma, Inc., High Point, NC (US)

(21) Appl. No.: 12/874,455

(22) Filed: Sep. 2, 2010

Related U.S. Application Data
(60) Provisional application No. 61/241,655, filed on Sep. 11, 2009.

(51) Int. Cl. A61K 9/20; A61K 31/5383; A61P 3/10; A61P 5/50; C07D 498/14

(52) U.S. Cl. 424/465; 514/229.8; 544/101

(57) ABSTRACT

The invention provides solid compositions comprising (S)-3-(4′-Cyano-biphenyl-4-yl)-2-{[(3S,7S)-3-[4-(3,4-dichloro-benzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid (OC-1) or a salt thereof and methods of making and using those compositions. The invention also provides the monohydrochloride salt of (S)-3-(4′-Cyano-biphenyl-4-yl)-2-{[(3S,7S)-3-[4-(3,4-dichloro-benzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid.

### CROSS-REFERENCE TO RELATED APPLICATIONS

This application claims the benefit of priority to U.S. Provisional Patent Application No. 61/241,655, filed Sep. 11, 2009.

### BACKGROUND OF THE INVENTION

Type 2 diabetes is a metabolic disorder where disease progression may be characterized by peripheral tissue insulin resistance, hyperglycemia, islet b-cell Vcompensation, hyperinsulinemia, dyslipidemia, increased liver gluconeogenesis and ultimate loss of b-cell mass and function. The pathophysiological consequences of aberrant glucose and lipid metabolism are toxicity to organs such as, but not limited to, the kidney, eye, peripheral neurons, vasculature and heart. Thus, there is a medical need for agents that may delay or prevent disease progression by improving glycemic control and b-cell mass and function.

Glucagon-like peptide-1 (GLP-1) is a member of the incretin family of neuroendocrine peptide hormones secreted from L-cells of the intestine in response to food ingestion. GLP-1 has multiple metabolic effects that are attractive for an antidiabetic agent. A key function of GLP-1 is to activate its receptor, GLP-1R, on the pancreatic b-cell to enhance glucose-dependent insulin secretion. Positive metabolic benefits of GLP-1 may include, but are not limited to, suppression of excessive glucagon production, decreased food intake, delayed gastric emptying, and improvement of b-cell 25 mass and function. The positive effects of GLP-1 on b-cell mass and function offers the hope that GLP-1-based therapies may delay early stage disease progression. In addition, a GLP-1 agonist could be useful in combination therapies such as with insulin in patients with type I diabetes. Unfortunately, the rapid proteolysis of GLP-1 into an inactive metabolite limits its use as a therapeutic agent.

Validation of GLP-1R agonists as a therapeutic modality was achieved by Exendin-4 (Byetta®, Amylin Pharmaceuticals, Inc.), a peptide GLP-1 receptor agonist recently approved for the treatment of type 2 diabetes. Dosing of Exendin-4 by subcutaneous administration lowers blood glucose and decreases HbA1c levels, which are important biomarker measurements for disease control. Still, a need exists in the art for an oral GLP-1 receptor agonist which provides glycemic control while offering the convenience of oral dosing.

GLP-1R belongs to the class B receptor sub-class of the G protein-coupled receptor (GPCR) superfamily that regulates many important physiological and pathophysiological processes. In addition to the seven transmembrane domains characteristic of all GPCR family members, class B GPCRs contain a relatively large N-terminal domain. It is believed the binding and activation of these receptors by relatively large natural peptide ligands require both the N-terminal domain and the transmembrane domain of the receptor. In particular, class B GPCRs have proven difficult for the identification of low molecular weight non-peptide agonist molecules. Because peptides, such as GLP-1, may lack sufficient oral bioavailability for consideration as oral drug agents, small molecule modulators of GLP-1R with oral bioavailability are highly desired.

### SUMMARY OF THE INVENTION

(S)-3-(4′-Cyano-biphenyl-4-yl)-2-{[(3S,7S)-3-[4-(3,4-dichloro-benzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, referred to herein as “OC-1”, is an agonist of GLP-1R. The preparation and pharmaceutical use of OC-1 and salts thereof is described in U.S. Pat. No. 7,727,983. OC-1 and salts thereof, however, may have very poor aqueous solubility. For example, the aqueous solubility of the hydrochloric acid salt of OC-1, increases at pH levels at or above 7 but it is only 0.0008 mg/mL at pH 6-7 where absorption by the body takes place. This poor aqueous solubility may correspond to poor absorption for OC-1 or salts thereof when administered orally. Thus, there is a need therefore to provide an oral dosage form of OC-1 or salts thereof with improved dissolution and/or absorption of OC-1 or salts thereof leading to improved oral bioavailability.

The invention provides solid compositions comprising OC-1 or a salt thereof and methods of making those compositions. The solid compositions may be in various oral dosage forms such as, but not limited to, capsules or tablets.

In various embodiments, the invention provides solid compositions comprising OC-1 or a salt thereof and at least one pharmaceutically acceptable basic excipient. In some embodiments, OC-1 or a salt thereof is present in its amorphous form.

In other embodiments, the invention provides solid compositions comprising at least one pharmaceutically acceptable basic excipient and an evaporation residue of OC-1 or a salt thereof. In further embodiments, the evaporation residue may further comprise at least one pharmaceutically acceptable polymeric stabilizing agent. In some embodiments, OC-1 or a salt thereof is present in the evaporation residue in its amorphous form.

The invention further provides methods of treating type 2 diabetes and high blood glucose levels by administering solid compositions of the invention.

The invention further provides a monohydrochloride salt of OC-1.

### BRIEF DESCRIPTION OF THE DRAWINGS

FIG. 1 is an exemplary XRD of the amorphous hydrochloric acid salt of OC-1 (1:1), as described herein.

FIG. 2 is an exemplary DSC of the amorphous hydrochloric acid salt of OC-1 (1:1), as described herein.

### DETAILED DESCRIPTION OF THE INVENTION

The invention provides solid compositions comprising OC-1 or a salt thereof and methods of making those compositions. The solid compositions may be in various oral dosage forms such as, but not limited to, capsules or tablets.

More particularly, in various embodiments, the invention provides solid compositions comprising OC-1 or a salt thereof and at least one pharmaceutically acceptable basic excipient. In some embodiments, OC-1 or a salt thereof is present in its amorphous form.

In other embodiments, the invention provides solid compositions comprising at least one pharmaceutically acceptable basic excipient and an evaporation residue of OC-1 or a salt thereof and may further comprise at least one pharmaceutically acceptable polymeric stabilizing agent. In some embodiments, OC-1 or a salt thereof is present in the evaporation residue in its amorphous form.

The invention further provides methods of treating type 2 diabetes and high blood glucose levels by administering solid compositions of the invention.

As used herein, the term “solid composition” refers to compositions that are, or may be made into, a solid pharmaceutical dosage form. By way of example only, in various exemplary embodiments, the solid compositions may be powders comprising amorphous OC-1 or a salt thereof and may further be in a dosage form suitable for oral administration to a subject, such as a capsule or tablet. In additional exemplary embodiments, the compositions may comprise amorphous OC-1 or a salt thereof mixed with other components described herein in a powder and may further be in a dosage form suitable for administration to a subject, such as a capsule or tablet.

As used herein, the term “OC-1 or salt thereof” refers to OC-1 or salts of OC-1. Salts of OC-1 are generally prepared by reacting the free base with a suitable organic or inorganic acid or by reacting the acid with a suitable organic or inorganic base. In various embodiments a salt of OC-1 is an acid addition salt of OC-1. In further embodiments, a salt of OC-1 is a hydrochloric acid salt of OC-1. In even further embodiments, a salt of OC-1 is a 1:1 hydrochloric acid salt of OC-1 (i.e., a monohydrochloride salt of OC-1). In any embodiment herein referring to “OC-1 or salt thereof,” a further embodiment may be to “amorphous OC-1 or an amorphous salt thereof.”

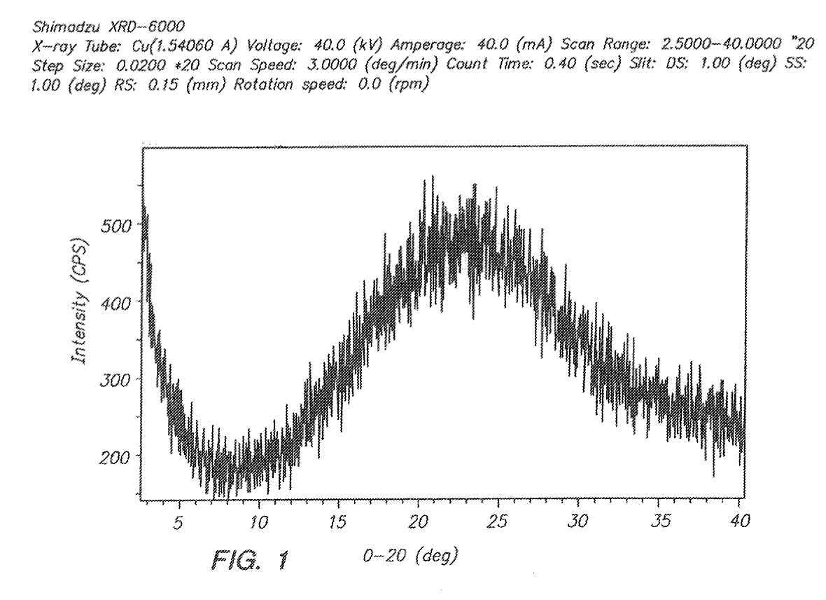

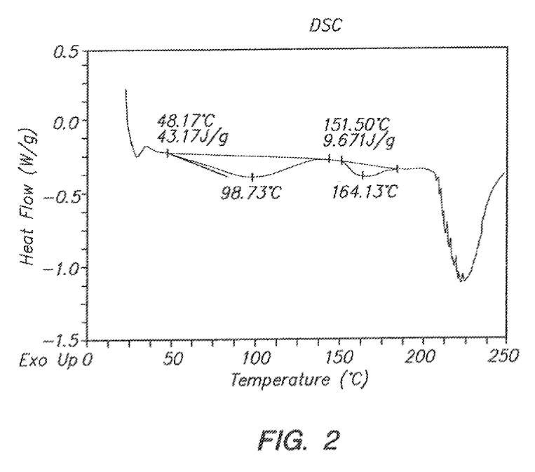

As used herein, the term “amorphous OC-1 or an amorphous salt thereof” refers to amorphous OC-1 or an amorphous salt of OC-1. In various embodiments, an amorphous salt of OC-1 may be an acid addition salt of OC-1. In further embodiments, an amorphous salt of OC-1 may be a hydrochloric acid salt of OC-1. In even further embodiments, a salt of OC-1 is a 1:1 hydrochloric acid salt of OC-1. The amorphous compound may be characterized by XRD or DSC. For example, an amorphous 1:1 hydrochloric acid salt of OC-1 may be, characterized by the XRD of FIG. 1 and/or DSC of FIG. 2, provided herein.

The amount of OC-1 or a salt thereof in the solid compositions of the invention may easily be determined by those of skill in the art. In various embodiments, OC-1 or a salt thereof may be present in a therapeutically effective amount. As used herein, the term “therapeutically effective amount” refers to an amount of OC-1 or salt thereof that elicits the biological or medicinal response in a tissue, system, or subject that is being sought by a researcher, veterinarian, medical doctor, patient or other clinician, which includes reduction or alleviation of the symptoms of the disease being treated. As used herein, the term “subject” includes, for example, horses, cows, sheep, pigs, mice, dogs, cats, and primates such as chimpanzees, gorillas, rhesus monkeys, and, humans. In one embodiment, a subject is a human. In another embodiment, a subject is a human in need of activation of GLP-1R.

When OC-1 is administered as a salt, references to the amount of active ingredient are to the free acid or free base form of the compound. That amount can, for example, be an amount sufficient to exhibit a detectable therapeutic effect, and can be determined by routine experimentation by those of skill in the art. The effect may include, for example, treatment of the conditions identified herein. The actual amount required, e.g. for treatment of any particular subject, will depend upon a variety of factors including the disorder being treated; its severity; the specific solid composition employed; the age, body weight, general health, gender, and diet of the subject; the mode of administration; the time of administration; the route of administration; the rate of excretion of the therapeutic agent; the duration of the treatment; any drugs used in combination or coincidental with the therapeutic agent; and other such factors well known to those skilled in the art. In various embodiments, for example, the solid composition may contain 1 mg or more of OC-1 in a given dosage, for example 5 mg or more, 10 mg or more, 20 mg or more, 40 mg or more, 50 mg or more, 100 mg or more, 200 mg or more, or 300 mg or more of amorphous OC-1 per dosage. In other embodiments, for example, the solid composition may contain less than 400 mg of amorphous OC-1 per dosage or less than 800 mg of amorphous OC-1 per dosage.

The invention further provides solid compositions comprising OC-1 or a salt thereof and at least one pharmaceutically acceptable basic excipient. In some embodiments, OC-1 or a salt thereof is present in its amorphous form.

As used herein and as known in the art, the term “pharmaceutically acceptable basic excipient” refers to any metal salt of an acid which demonstrates basic properties in either the Bronsted or Lewis sense, which includes those salts where all protons have been replaced with a mono or polyvalent metal ion and extends to those metal salts of acids which contain a proton but demonstrate a pH of 7 or greater. Many such salts, particularly those of inorganic acids and many organic acids, may be water soluble, but water solubility is not a limiting factor in selecting a basic excipient. Metal salts of surfactants, whether water-soluble or water dispersible, are also within the scope of the basic excipients as defined herein. The pharmaceutically acceptable basic excipients of the disclosure are generally regarded as safe, at least in the dosage amounts used.

Pharmaceutically acceptable basic excipients include, but are not limited to, any of the numerous salts of inorganic acids, short chain mono, di or tri carboxylic acids, or salts of the various long-chain fatty acids or sulfonated fatty acids and alcohols and related surfactants. Selected salts should be inert in the sense that they themselves would not be expected or intended to demonstrate any deleterious or untoward pharmacological effects on the host to which these dosage forms are applied.

Pharmaceutically acceptable basic excipients of inorganic acids include, for example: basic alkali metal salts of phosphoric acid, such as disodium phosphate, dipotassium phosphate, and calcium phosphate; basic alkali metal salts of orthophosphate, hypophosphate, and pyrophosphate, such as the di and trisodium forms of orthophosphate, the di and tripotassium orthophosphates, magnesium orthophosphate, and magnesium pyrophosphate, sodium or potassium hypophosphate, sodium or potassium pyrophosphate, calcium hypophosphate and calcium orthophosphate, including the mono, di and tri calcium forms, calcium pyrophosphate, and mixed alkali metal salts of these various phosphates; alkali metal salts of nitric acids, such as sodium nitrate, potassium nitrate, calcium nitrate, and magnesium nitrate; alkali metal salts of sulfuric acid, such a sodium sulfate, potassium sulfate, magnesium sulfate, and calcium sulfate; and alkali metal salts of boric acid, such as sodium borate or potassium borate.

Pharmaceutically acceptable basic excipients further include basic alkali metal salts of various mono, di or tri carboxylic acids, for example, the alkali metal salts of carbonic acid, such as sodium bicarbonate, sodium carbonate, potassium carbonate, potassium bicarbonate, sodium potassium carbonate, magnesium carbonate or calcium carbonate may be used herein.

Pharmaceutically acceptable basic excipients further include alkaline metal salts of organic acids, such as formic acid, acetic acid, propionic acid, glycolic acid, lactic acid, pyruvic acid, oxalic acid, malonic acid, succinic acid, malic acid, maleic acid, fumaric acid, tartaric acid, benzoic acid, cinnammic acid, and mandelic acid.

In at least one embodiment, the at least one pharmaceutically acceptable basic excipient used may be chosen from trisodium phosphate, potassium carbonate, sodium carbonate, sodium bicarbonate, or a mixture thereof. In another embodiment, the at least one pharmaceutically acceptable basic excipient used may be a mixture of sodium carbonate and sodium bicarbonate. In another embodiment, the at least one pharmaceutically acceptable basic excipient may comprise sodium carbonate.

In various embodiments, the at least one pharmaceutically acceptable basic excipient may be present in a solid composition in an amount such that the ratio of pharmaceutically acceptable basic excipient to OC-1 or a salt thereof may range from 1:2 to 5:1, for example, the ratio may be 1:1, 3:1, or 4:1. In an embodiment, the ratio of pharmaceutically acceptable basic excipient to OC-1 or a salt thereof may range from 1:2 to 2:1. The amount of at least one pharmaceutically acceptable basic excipient may vary depending, in part, upon the specific solid composition, including the amount of OC-1 or a salt thereof. The amount of at least one pharmaceutically acceptable basic excipient may also vary, in part, depending upon the particular basic excipient chosen. For example, the amounts of basic excipients used that are strong bases, i.e., have a low pK_{b} values, may be smaller than the amounts used for those basic excipients that are weak bases, i.e., have high pK_{b} values.

In a further aspect of the invention, the solid composition may comprise at least one pharmaceutically acceptable basic excipient and an evaporation residue of OC-1 or a salt thereof. In some embodiments, OC-1 or a salt thereof is present in its amorphous form. In various embodiments, the at least one pharmaceutically acceptable basic excipient may be present in the evaporation residue. In additional embodiments, the evaporation residue may further comprise at least one pharmaceutically acceptable polymeric stabilizing agent.

As used herein, the term “evaporation residue” refers to the solids remaining after removal of solvent from a solution and/or suspension of OC-1 or a salt thereof, alone or in combination with other components.

Pharmaceutically acceptable polymeric stabilizing agents include, but are not limited to, polyvinylpyrrolidone (PVP), hydroxypropylmethyl cellulose acetate succinate (HPMCAS), hydroxypropylmethyl cellulose phthalate (HPMCP), hydroxypropylmethyl cellulose (HPMC), poloxamers, hydroxypropyl methyl cellulose acetate, hydroxypropyl methyl cellulose, hydroxypropyl cellulose, and hydroxyethyl cellulose acetate, polyacrylates, methyl acrylatemethacrylic acid copolymers, ethyl acrylatemethacrylic acid copolymers, cellulose acetate phthalate, cellulose acetate trimellitate, carboxymethyl ethyl cellulose and mixtures thereof.

In at least one embodiment, the at least one pharmaceutically acceptable polymeric stabilizing agent may be HPMCAS or PVP. In another embodiment of the invention, the at least one pharmaceutically acceptable polymeric stabilizing agent may be HPMCAS. In another embodiment of the invention, the at least one pharmaceutically acceptable polymeric stabilizing agent may be PVP.

In various embodiments, the amount of at least one pharmaceutically acceptable polymeric stabilizing agent present in a solid composition may be present in an amount such that the ratio of pharmaceutically acceptable polymeric stabilizing agent to OC-1 or salt thereof may range from 1:200 to 4:1, for example, the ratio may be 1:2 or 1:1. In another embodiment, the ratio of pharmaceutically acceptable polymeric stabilizing agent to OC-1 or salt thereof may range from 1:1 to 4:1, or from 1:2 to 2:1. The amount of at least one pharmaceutically acceptable polymeric stabilizing agent may vary depending, in part, upon the specific solid composition, including the amount of OC-1 or salt thereof.

In various embodiments of the invention, the solid composition comprises an evaporation residue of OC-1 or a salt thereof and optionally at least one pharmaceutically acceptable polymeric stabilizing agent and/or at least one pharmaceutically acceptable basic excipient, which may be formed by mixing OC-1 or a salt thereof and optionally at least one pharmaceutically acceptable polymeric stabilizing agent and/or at least one pharmaceutically acceptable basic excipient in at least one solvent to form a solution or suspension and removing the solvent from the solution or suspension to form an evaporation residue. In some embodiments, OC-1 or a salt thereof is present in the evaporation residue in its amorphous form.

Acceptable solvents include, but are not limited to, water or other polar solvents such as alcohols, for example ethanol and isopropanol, ketones, for example acetone, and mixtures thereof. In various embodiments, the solvent may be chosen from water, ethanol, and acetone. In a further embodiments, the suspension may be a nanosuspension of OC-1 or a salt thereof in the solvent. Nanosuspensions may be prepared by, for example, milling. precipitation, homogenization or any combination of any of these methods. For example, OC-1 or a salt thereof and at least one pharmaceutically acceptable polymeric stabilizer and a wetting agent, for example pluronic, may be suspended in a solvent and milled to produce a nanosuspension. The nanosuspension may then be filtered to obtain the desired particle size distribution, for example through a 0.45 micron or 1.2 micron syringe filter.

Removal of the solvent from the solution or suspension may, in various embodiments, comprise spray drying the solution or suspension to form a powder. In other exemplary embodiments, the solution may be removed by evaporation, for example by using a rotovap or a flat-bed dryer to form an evaporation residue.

In a further embodiment, the spray drying step may comprise spraying the solution or suspension onto a solid pharmaceutically acceptable carrier to form a mixture. As used herein and as known in the art, the term “pharmaceutically acceptable carrier” refers to pharmaceutically acceptable basic excipients, as described herein, pharmaceutically acceptable inert carriers, and/or mixtures thereof. As used herein and as known in the art, the term “pharmaceutically acceptable inert carriers” refers to those inorganic and organic carriers that are physiologically harmless and are not basic excipients. In addition to the pharmaceutically acceptable basic excipients listed above, soild pharmaceutically acceptable carriers include, but are not limited to edible carbohydrates, for example, starches, lactose, sucrose, glucose, and mannitol, silicic acid, calcium carbonate, calcium phosphate, sodium phosphate, crospovidone, and kaolin.

In other embodiments, the solid composition may be formed by mixing the at least one pharmaceutically acceptable basic excipient with a powdered pharmaceutically acceptable carrier onto which the solution or suspension containing OC-1 or a salt thereof and optionally at least one pharmaceutically acceptable polymeric stabilizing agent is sprayed. The evaporation residue is formed on and mixed with the powdered pharmaceutically acceptable carrier, which may be premixed with the pharmaceutally acceptable basic excipient or mixed after the spry drying step.

In yet other embodiments, the at least one pharmaceutically acceptable basic excipient may be mixed with an evaporation residue of OC-1 or a salt thereof and optionally at least one pharmaceutically acceptable polymeric stabilizing agent.

The solid compositions of the invention may further comprise at least one water-soluble surfactant. The at least one water-soluble surfactant of the invention may be chosen from, but is not limited to, sulfuric acid alkyl ester salts, such as sodium lauryl sulfate; bile acid salts, such as sodium taurocholate and sodium glycocholate; propylene glycol fatty acid mono- or diesters, such as those sold under the trade name Miglyol® 840 by Sasol Olefins and Surfactants of Huston, Tex., USA; polyethylene glycol fatty acid esters, such as polyethylene glycol monooleate and polyethylene glycol monostearate; polysorbates, such as polyoxyethylene sorbitan fatty acid esters sold under the trade names TWEEN® 20, TWEEN 40®, and TWEEN® 80 by Spectrum Chemicals of Gardena, Calif., USA; polyoxyethylene-polyoxypropylene copolymer and block copolymer surfactants, such as poloxamer 188, poloxamer 235, poloxamer 404, and poloxamer 407 and those sold under the trade names PLURONIC® F87, PLURONIC® F127, PLURONIC® F68, PLURONIC® L44, PLURONIC® P123, and PLURONIC® P85 by BASF of BASF of Mt. Olive, N.J., USA; polyoxyethylene derivatives of natural oils and waxes, such as polyoxyethylene castor oil and polyoxyethylene hydrogenated castor oil, for example those sold under the trade names CREMOPHOR® RH40 and CREMOPHOR® EL by BASF of BASF of Mt. Olive, N.J., USA; and sorbitan fatty acid esters, such as sorbitan monooleate, sorbitan monostearate, sorbitan monopalmitate, sorbitan monolaurate, and sorbitan monocaprylate, sold under the trade names SPAN® 80, SPAN® 60, SPAN® 40, SPAN® 20, and SEFSOL® 418, respectively, by Croda International PLC of Goole, U.K. The selection and amount of the at least one water soluble surfactant may be based, in part, upon its compatibility with the other ingredients in the composition, the amount of OC-1 or a salt thereof, and consideration that it is not deleterious to the recipient thereof.

In various embodiments, the solid composition may comprise OC-1 or a salt thereof, at least one pharmaceutically acceptable basic excipient, and at least one water-soluble surfactant. In some embodiments, OC-1 or a salt thereof is in its amorphous form.

In another embodiment, the solid composition may comprise an evaporation residue of OC-1 or a salt thereof, at least one pharmaceutically acceptable basic excipient, at least one pharmaceutically acceptable polymeric stabilizing agent, and at least one water-soluble surfactant. In some embodiments, OC-1 or a salt thereof is present in the evaporation residue in its amorphous form.

The solid compositions of the invention may further comprise at least one additional pharmaceutical ingredient. As used herein, the term “additional pharmaceutical ingredient” is intended to mean a component or excipient other than powdered pharmaceutically acceptable carriers. Non-limiting examples of additional ingredients include:

a) glidants and lubricants, such as colloidal silica, talc, magnesium stearate, calcium stearate, stearic acid, solid polyethylene glycol, sodium oleate, sodium stearate, sodium benzoate, sodium acetate, sodium chloride, sodium stearyl furamate, and sodium lauryl sulfate;

b) disintegrating and solubilizing agents, such as agar-agar, calcium carbonate, sodium carbonate, croscarmellose sodium, starches, pregelatinized starches, sodium starch glycolate, crospovidone, methyl cellulose, agar, bentonite, xanthan gum, alginic acid, and certain silicates;

c) binding agents, such as starches, gelatin, natural sugars, for example, glucose, sucrose, or beta-lactose, corn sweeteners, natural and synthetic gums, for example acacia, tragacanth, or sodium alginates, acadia mucilage, carboxymethylcellulose, microcrystalline cellulose, polyethylene glycol, polyvinylpyrrolidinone, and waxes;

d) solution retarding agents, such as polymers, for example biodegradable polymers such as polylactic acid, polyepsilon caprolactone, polyhydroxy butyric acid, polyorthoesters, polyacetals, polydihydropyrans, polycyanoacrylates, and cross-linked or amphipathic block copolymers of hydrogelsparaffin, and wax, for example, paraffin;

e) resorption accelerating agents, such as quaternary ammonium compounds;

f) absorption agents, such as quaternary ammonium compounds, bentonite, kaolin, or dicalcium phosphate;

g) wetting agents and humectants, such as cetyl alcohol and glycerol monostearate; and

h) fillers, such as anhydrous lactose, microcrystalline cellulose, mannitol, calcium phosphate, pregelatinized starch, and sucrose.

Pharmaceutically acceptable adjuvants known in the pharmaceutical formulation art may also be used as additional pharmaceutical ingredients in the solid compositions of the invention. These include, but are not limited to, preserving, suspending, sweetening, flavoring, coloring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. If desired, a solid composition of the invention may also contain minor amounts of auxiliary substances such as wetting or emulsifying agents, pH buffering agents, antioxidants, and the like, such as, for example, vitamin E TPGS, fumed silica, citric acid, sorbitan monolaurate, triethanolamine oleate, butylalted hydroxytoluene, etc.

It is within the ability of one of skill in the art to select the at least one additional ingredient and the amount of said additional ingredient. The selection and amount of the at least one additional ingredient may be based, in part, upon its compatibility with the other ingredients in the formulation, the amount of OC-1 or salt thereof, and consideration that it is not deleterious to the recipient thereof.

The invention further relates to the solid compositions described herein in a form for oral administration as discrete units, such as capsules or tablets. Preparation of the solid compositions in forms intended for oral administration is within the ability of one skilled in the art, including the selection of pharmaceutically acceptable additional ingredients from the groups listed above in order to provide pharmaceutically elegant and palatable preparations. For example, the solid compositions of the invention may be prepared by methods known in the pharmaceutical formulation art, for example, see Remington's Pharmaceutical Sciences, 18th Ed., (Mack Publishing Company, Easton, Pa., 1990).

In various embodiments, capsules may be prepared by, for example, preparing a powder mixture comprising OC-1 or a salt thereof and at least one pharmaceutically acceptable basic excipient and encapsulating the powder with gelatin or some other appropriate shell material. Additional ingredients, such as those set forth above and including glidants and lubricants and disintegrating and solubilizing agents, may be added to the powder before the encapsulation.

In various other embodiments, tablets may be prepared by, for example, preparing powder mixture and pressing the mixture into tablets. Additional ingredients, such as those set forth above and including glidants and lubricants, disintegrating and solubilizing agents, binders, solution retardants, and absorption agents, may be added to the powder before pressing into tablets. The powder mixture may be wet-granulated with a binder such as syrup, starch paste, acadia mucilage or solutions of cellulosic or polymeric materials, and forcing through a screen. Or, in other embodiments, the powder mixture may be run through the tablet machine, producing slugs broken into granules. Then granules may be lubricated and then compressed into tablets. In a further embodiment, the powder mixture may be compressed directly into tablets without granulation or slugging.

In various embodiments, tablets of the invention may be multilayer tablets. For example, OC-1 or a salt thereof mixed with at least one pharmaceutically acceptable stabilizing agent, at least one water-soluble surfactant, or at least one additional ingredient may be compressed to form one layer of a multilayer tablet. At least one pharmaceutically acceptable basic excipient may be compressed to form one layer of a multilayer tablet. In at least one embodiment, the OC-1 layer and basic excipient layer may be combined to form a multilayer tablet. In a further embodiment, the OC-1 layer and basic excipient layer may be separated by an additional layer comprising additional ingredients.

The tablets of the invention may be uncoated or coated. In various embodiments, tablets may be coated with a clear or opaque protective coating, which may for example, consist of a sealing coat of shellac, a coating of sugar or polymeric material, and a polish coating of wax. In various embodiments, tablets may be coated to delay disintegration and absorption in the gastrointestinal tract and thereby provide a sustained action over a longer period. Such coatings may comprise glyceryl monostearate or glyceryl distearate. Additionally, dyestuffs can be added to these coatings to distinguish different unit dosages.

The solid compositions of the invention may exhibit improved bioavailability of OC-1 or salts thereof upon administration to a subject relative to solid compositions that do not include OC-1 or a salt thereof and at least one pharmaceutically acceptable basic excipient.

As used herein, the term “improved bioavailability” means that the bioavailability of OC-1 delivered in the solid composition of the invention is increased and may be approximately at least double, relative to the bioavailability of conventional compositions, for example at least three times, at least five times, or at least ten times that of conventional compositions. It is within the ability of one of skill in the art to determine the bioavailability of a compound or composition using methods generally accepted in the art. For example, the maximum concentration (Cₘₐₓ) of OC-1 in plasma or the overall amount of OC-1 in plasma after a dosage, e.g., area-under-the-curve (AUC), may be used for the comparison. These pharmacokinetic measurements may be determined by conventional techniques. For example, in various embodiments, the concentration of OC-1 in plasma may be determined by a LC-MS/MS assay following a protein precipitation step with acetonitrile. In additional embodiments, pharmacokinetic analysis may be performed using the WinNonlin™ software program, which is available from Pharsight, Inc. of Mountain View, Calif., USA. The area under the plasma concentration-time curve (AUC₀₋ₜ) may be calculated from the first time point (0 min) up to the last time point with measurable drug concentration. The AUC_{0-inf} may be calculated as the sum of AUC₀₋ₜ and Cpred/λz, where Cpred was the predicted concentration at the time of the last quantifiable concentration.

In various embodiments, improvements in bioavailability may be based, in part, upon the selection of and amount of at least one pharmaceutically acceptable basic excipient and optional at least one pharmaceutically acceptable stabilizing agent. For example, if a strong base is used or a large amount of the basic excipient, bioavailability may increase more greatly.

The solid compositions of the invention may also exhibit chemical stability. As used herein, the terms “stability,” “stable,” and variations thereof, are intended to mean that less than 10% of the OC-1 or a salt thereof in the composition decomposes over a period of 1 to 4 weeks at 40° C. and 75% relative humidity. Stability may also be tested under the influence of a variety of other conditions. In various embodiments, for example, less than 8%, less than 6%, less than 4%, less than 2%, or less than 1% of the OC-1 or a salt thereof may decompose. It is within the ability of one of skill in the art to determine the stability of a compound or composition using methods generally accepted in the art. For example, the amount of OC-1 or a salt thereof or of another ingredient in the composition decomposed may be measured by any suitable method, e.g., HPLC. Decomposition is typically a chemical process made up of at least one reaction, such as oxidation, reduction, or hydrolysis, which results in a chemical change in the decomposing substance resulting in the generation of one or more new chemical compounds. As used herein the term “impurity” means any such new compound that is present in the composition in an amount less than 10 wt % of the composition, for example less than 5 wt %, or less than 1 wt % of the composition. In other embodiments, stability may be determined by other characteristics, such as appearance.

The invention further relates to methods for the treatment of type 2 diabetes or high blood glucose levels using any one of the solid compositions of the invention. For example, invention relates to methods for the treatment of type 2 diabetes or high blood glucose levels the method comprising administering to a subject a solid composition comprising a therapeutically effective amount of OC-1 or a salt thereof.

The invention also relates to a method of lowering blood glucose concentration in a subject comprising administering any one of the solid compositions of the invention. For example, the invention relates to a method of lowering blood glucose concentration in a subject comprising administering to a subject a solid composition comprising a therapeutically effective amount of OC-1 or a salt thereof. In a further embodiment, the method lowers fasting blood glucose concentration in a subject. In another embodiment, the method lowers postprandial blood glucose concentration in a subject. In another embodiment, the subject is suffering from type 2 diabetes.

The invention also relates to a method of stimulating insulin secretion in a subject comprising administering any one of the solid compositions of the invention. For example, the invention relates to a method of stimulating insulin secretion in a subject comprising administering to a subject a solid composition comprising a therapeutically effective amount of OC-1 or a salt thereof. In various embodiments, the subject is suffering from type 2 diabetes.

The solid compositions administered in these methods of the invention are the same in the various embodiments as those discussed above. Thus, in an embodiment of any of the methods of treatment, methods of lowering blood glucose concentration, or methods of stimulating insulin secretion above, a solid composition may be administered wherein the solid composition comprises OC-1 or a salt thereof and at least one pharmaceutically acceptable basic excipient. In some embodiments, OC-1 or a salt thereof is present in its amorphous form.

In another embodiment of any of the methods of treatment above, a solid composition may be administered wherein the solid composition comprises at least one pharmaceutically acceptable basic excipient and an evaporation residue of OC-1 or a salt thereof. In a further embodiment, the evaporation residue may further comprise at least one pharmaceutically acceptable polymeric stabilizing agent. In some embodiments, OC-1 or a salt thereof is present in the evaporation residue in its amorphous form. In some embodiments, the salt of OC-1 is an acid addition salt of OC-1. In further embodiments, the salt of OC-1 is a hydrochloric acid salt of OC-1. In even further embodiments, the salt of OC-1 is a 1:1 hydrochloric acid salt of OC-1.

### EXAMPLES

The following examples are not intended to be limiting of the invention as claimed.

The following commercially available materials were used in the examples below:

HPMCAS polymeric binders (AQOAT, MG and LG type) are available from Shinetsu Chemical Industries Co., Ltd. of Tokyo, Japan;

Avicel PH101, microcrystalline cellulose, is available from FMC Biopolymer of Newark Del., USA;

Cabosil, fumed silica, is available from Cabot of Tuscola, Ill., USA;

Plasdone K29-32, polyvinylpyrrolidone, is available from Spectrum Chemicals of Gardena, Calif., USA;

Pluronic F127, a poloxamer surfactant, is available from BASF of Mt. Olive, N.J., USA; and

Polysorbate 80 (Tween 80) surfactant is available from Spectrum Chemicals of Gardena, Calif., USA.

### Example A

Jetmilled micronized HCl salt of OC-1 (3.39 g) (Particle size distribution measured using laser light defraction in oil dispersion (10% 0.77 micrometers, 50% 18.23 micrometers, 90%, 111.77 micrometers)) was thoroughly blended with 2.46 g of Avicel PH101, 2.46 g of lactose, 0.05 g of Cabosil, 0.60 g of croscarmellose sodium and 0.05 g of magnesium stearate. The resulting mixture was filled into size 0 hard gelatin capsules. Each capsule contained 300 mg of powder and 100 mg of HCl salt of OC-1.

### Example B

3.36 g of HCl salt of OC-1, 0.42 g of Tween 80, 0.42 g of vitamin E TPGS and 0.02 g of Plasdone K29-32 were dissolved into 60 mL of ethanol. The solution was spray dried onto a mixture of 1.18 g of Avicel PH101, 1.18 g of lactose, and 0.42 g of crospovidone using a fluidized bed granulation apparatus to obtain mixture of fine powder and small granules. 5.99 g of mixture was thoroughly blended with 0.27 g of Avicel PH101, 0.27 g of lactose, 0.20 g of crospovidone, and 0.04 g of magnesium stearate. The resulting mixture was compressed into tablets using SC-2 single station tablet press from Key International; each tablet had hardness of 8-12 Kp. Each tablet weighed 250 mg and contained 100 mg of HCl salt of OC-1.

### Example C

5.60 g of HCl salt of OC-1 and 0.05 g of Plasdone K29-32 were dissolved into 75 mL of ethanol. The solution was spray dried onto a mixture of 2.52 g of Avicel PH101, 2.52 g of lactose, and 0.50 g of crospovidone using a fluidized bed granulation apparatus to obtain mixture of fine powder and small granules. 7.71 g of mixture was thoroughly blended with 0.17 g of Avicel PH101, 0.69 g of lactose, and 0.05 g of magnesium stearate. The resulting mixture was compressed into tablets using SC-2 single station tablet press from Key International; each tablet had hardness of 8-12 Kp. Each tablet weighed 250 mg and contained 100 mg of HCl salt of OC-1.

### Example D

5.60 g of HCl salt of OC-1, 0.45 g of Tween 80 and 0.05 g of Plasdone K29-32 were dissolved into 75 mL of ethanol. The solution was spray dried onto a mixture of 2.52 g of Avicel PH101, 2.52 g of lactose, and 0.50 g of crospovidone using a fluidized bed granulation apparatus to obtain mixture of fine powder and small granules. 7.71 g of mixture was thoroughly blended with 0.17 g of Avicel PH101, 0.69 g of lactose, 0.24 g of crospovidone, and 0.05 g of magnesium stearate. The resulting mixture was compressed into tablets using SC-2 single station tablet press from Key International; each tablet had hardness of 8-12 Kp. Each tablet weighed 275 mg and contained 100 mg of HCl salt of OC-1.

### Example E

11.2 g of HCl salt of OC-1 was suspended in a solution of 1.0 g of Pluronic F127, 1.0 g of Tween 80, and 0.1 g of Plasdone K29-32 in 100 mL of water. The mixture was milled in a mill (DYNO-MILL Multilab) to produce a nanosuspension. The solution was filtered through a 1.2 micron syringe filter. The nanosuspension was assayed to contain 70 mg/mL OC-1. 58 mL of the nanosuspension was spray dried onto 3.00 g of Avicel PH101, 3.00 g of lactose, and 1.20 g of crospovidone using a fluidized bed granulation apparatus to obtain mixture of fine powder and small granules. 2.21 g of mixture was thoroughly blended with 0.16 of microcrystalline cellulose, 0.16 g of lactose, 0.07 g of crospovidone and 0.01 g of magnesium stearate, and the mixture was compressed into tablets using SC-2 single station tablet press from Key International; each tablet had hardness of 8-12 Kp. Each tablet weighed 300 mg and contained 100 mg of HCl salt of OC-1.

### Example F

2.24 g of HCl salt of OC-1 was dissolved in 20 mL of ethanol. 4.03 g of Plasdone K29-32 and 0.19 g of sodium lauryl sulfate were added to the solution and stirred for 3 minutes. The ethanol was evaporated in a rotavapor under vacuum to obtain dry powder. The powder was grinded with pestle in a mortal and passed through a #30 size mesh screen. The screened powder was thoroughly blended with 0.76 g of Avicel PH101 and 0.38 g of croscarmellose sodium. The resulting mixture was filled into size 1 hard gelatin capsules. Each capsule contained 152 mg of powder and 40 mg of HCl salt of OC-1.

### Example G

11.2 g of HCl salt of OC-1 was suspended in a solution of 1.0 g of Pluronic F127, 1.0 g of Tween 80, and 0.5 g of Plasdone K29-32 in 100 mL of water. The mixture was milled in a mill (DYNO-MILL Multilab) to produce a nanosuspension. The solution was filtered through a 1.2 micron syringe filter. The nanosuspension was assayed to contain 70 mg/mL OC-1. 58 mL of the nanosuspension was spray dried onto 3.00 g of Avicel PH101, 3.00 g of lactose, and 1.20 g of crospovidone using a fluidized bed granulation apparatus to obtain mixture of fine powder and small granules. 2.21 g of mixture was thoroughly blended with 0.16 of microcrystalline cellulose, 0.16 g of lactose, 0.07 g of crospovidone and 0.01 g of magnesium stearate, and the mixture was compressed into tablets using SC-2 single station tablet press from Key International; each tablet had hardness of 8-12 Kp. Each tablet weighed 300 mg and contained 100 mg of HCl salt of OC-1.

### Example H

0.60 g of Tween 80 and 0.06 g of HPMC (E3) were dissolved into 2 ml of water. The solution was dripped onto a mixture of 3.39 g of HCl salt of OC-1, 2.52 g of Avicel PH101, 2.52 g of lactose, and 0.84 g of croscarmellose sodium using a mixer apparatus to obtain small wet granules. The wet granules were dried for 4 hours at 40° C. in an oven and at room temperature overnight. The granules (8.27 g) were thoroughly blended with 0.87 g of Avicel PH101, 0.51 g of croscarmellose sodium, and 0.04 g of magnesium stearate. The resulting mixture was put inside a size 0 hard gelatin capsule. Each capsule contained 400 mg of powder and 100 mg of OC-1.

### Example I

2.24 g of HCl salt of OC-1 was dissolved in 20 mL of ethanol, 4.03 g of Plasdone K29-32 and 0.19 g of sodium lauryl sulfate was added to the solution and the ethanol was evaporated using ratovapor. The dried material was grinded using mortel and pestel and passed through #30 size mesh screen. 6.44 g of the mixture was thoroughly blended with 0.38 g of croscarmellose sodium, and 0.76 g of Avicel PH101. The resulting mixture was put inside a size 0 hard gelatin capsule. Each capsule contained 152 mg of powder and 40 mg of OC-1.

### Example J

5.6 g of HCl salt of OC-1, 5.0 g of AQOAT, MG type, and 0.5 g of Tween 80 were dissolved in 50 mL of acetone. The solution was sprayed dried in a spray dryer (Niro SDMicro spray drier, glass drying chamber, and filter housing; single pass nitrogen gass, 0.5 mm liquid insert, single point collection, at 1.0 bar. Inlet temperature between 70 and 80° C. for acetone) and dried to obtain fine powder. 1.33 g of the powder was thoroughly blended with 0.36 g of crospovidone, 0.26 g of Avicel PH101, 0.30 g of corn starch, 0.30 g pregelatinized starch, and 0.12 g of sodium lauryl sulfate. The powder was compressed in a tablet press, milled, and passed through a #40 mesh screen. The powder was then blended with 0.30 g of crospovidone, 0.26 g of Avicel PH101, 0.27 g of pregelatinized starch, 0.30 g of corn starch, 0.06 g of Cabosil, and 0.03 g of magnesium stearate. The resulting mixture was compressed into tablets using SC-2 single station tablet press from Key International; each tablet had hardness of 8-12 Kp. Each tablet weighed 650 mg and contained 100 mg of HCl salt of OC-1.

### Example K

11.2 g of HCl salt of OC-1 was suspended in a solution of 1.0 g of Pluronic F127, 1.0 g of Tween 80, and 0.5 g of Plasdone K29-32 in 100 mL of water. The suspension was milled in a mill (DYNO-MILL Multilab) to produce nanosuspension. The nanosuspension was filtered through 1.2 micron syringe filter and assayed.

### Example 1

11.2 g of HCl salt of OC-1 was suspended in a solution of 1.0 g of Pluronic F127, 1.0 g of Tween 80, and 0.5 g of Plasdone K29-32 in 100 mL of water. The mixture was milled in a mill (DYNO-MILL Multilab) to produce a nanosuspension. The nanosuspension was filtered through a 1.2 micron syringe filter. The nanosuspension was assayed to contain 75 mg/mL HCl salt of OC-1. 100 mL of the solution was sprayed onto 5.90 g of Avicel PH101, 5.90 g of lactose, and 1.44 g of crospovidone using a fluidized bed granulation apparatus and dried to obtain mixture of fine powder and small granules. 2.49 g of mixture was thoroughly blended with 0.38 of Avicel PH101, 0.13 g of pregelatinized starch, 0.21 g of crospovidone, and 0.02 g of magnesium stearate. 327 mg of the mixture was blend with 100 mg of potassium carbonate and filled into size 0 hard gelatin capsules. Each capsule had 427 mg of material and contained 100 mg of HCl salt of OC-1.

### Example 2

3.36 g of HCl salt of OC-1, 0.42 g of Tween 80, 0.42 g of vitamin E TPGS and 0.02 g of Plasdone K29-32 were dissolved into 60 mL of ethanol. The solution was spray dried onto a mixture of 1.18 g of Avicel PH101, 1.18 g of lactose, and 0.42 g of crospovidone using a fluidized bed granulation apparatus to obtain mixture of fine powder and small granules. 5.99 g of mixture was thoroughly blended with 0.27 g of Avicel PH101, 0.27 g of lactose, 0.20 g of crospovidone, and 0.04 g of magnesium stearate. 2.50 grams of the blend was mixed with 1.0 grams of potassium carbonate. The resulting mixture was compressed into tablets using SC-2 single station tablet press from Key International; each tablet had hardness of 8-12 Kp. Each tablet weighed 350 mg and contained 100 mg of HCl salt of OC-1.

### Example 3

11.20 g of HCl salt of OC-1 was suspended in a solution of 1.0 g of Pluronic F127, 1.0 g of Tween 80, and 0.5 g of Plasdone K29-32 in 100 ml of water. The mixture was milled in a mill (DYNO-MILL Multilab) to produce nanosuspension. The nanosuspension was filtered through a 1.2 micron syringe filter. The nanosuspension was assayed to contain 75 mg/ml HCl salt of OC-1. The solution was sprayed onto 5.90 g of Avicel PH101, 5.90 g of lactose, and 1.44 g of crospovidone using a fluidized bed granulation apparatus and dried to obtain mixture of fine powder and small granules. 2.49 g of the powder was thoroughly blended with 0.21 g of crospovidone, 0.38 g of Avicel PH101, 0.04 g of Cabosil, 0.12 g of Pregelatinized starch and 0.02 g of magnesium stearate. The resulting mixture was compressed into tablets using SC-2 single station tablet press from Key International; each tablet had hardness of 8-12 Kp. Each tablet weighed 327 mg and contained 100 mg of HCl salt of OC-1.

### Example 4

3.36 g of HCl salt of OC-1, 0.42 g of Tween 80, 0.42 g of vitamin E TPGS and 0.02 g of Plasdone K29-32 were dissolved into 60 mL of ethanol. The solution was spray dried onto a mixture of 1.18 g of Avicel PH101, 1.18 g of lactose, and 0.42 g of crospovidone using a fluidized bed granulation apparatus to obtain mixture of fine powder and small granules. 5.99 g of mixture was thoroughly blended with 0.27 g of Avicel PH101, 0.27 g of lactose, 0.14 g of pregelatinized starch, 0.20 g of crospovidone, 0.03 g of Cabosil, 0.06 g of corn starch and 0.04 g of magnesium stearate. 2.50 grams of the blend was mixed with 1.0 grams of potassium carbonate. The resulting mixture was put inside a size 0 hard gelatin capsule. Each capsule contained 380 mg of powder and 100 mg of OC-1.

### Example 5

4.48 g of HCl salt of OC-1, 0.02 g of HPMC (E3), 0.56 g of Tween 80, and 0.56 g of vitamin E TPGS were dissolved into 20 ml of acetone. The solution was sprayed onto 1.58 g of Avicel PH101, 1.58 g of lactose, and 0.56 g of crospovidone using a fluidized bed granulation apparatus and dried to obtain mixture of fine powder and small granules. 2.51 g of mixture was thoroughly blended with 0.08 g of Avicel PH101, 0.08 g of lactose, 0.08 g of crospovidone, and 0.01 g of magnesium stearate. The resulting mixture was compressed into tablets using SC-2 single station tablet press from Key International; each tablet had hardness of 8-12 Kp. Each tablet weighed 275 mg and contained 100 mg of HCl salt of OC-1.

### Example 6

3.36 g of HCl salt of OC-1, 0.42 g of Tween 80, 0.42 g of vitamin E TPGS and 0.02 g of Plasdone K29-32 were dissolved into 60 mL of ethanol. The solution was spray dried onto a mixture of 1.18 g of Avicel PH101, 1.18 g of lactose, and 0.42 g of crospovidone using a fluidized bed granulation apparatus to obtain mixture of fine powder and small granules. 5.99 g of mixture was thoroughly blended with 0.27 g of Avicel PH101, 0.27 g of lactose, 0.14 g of pregelatinized starch, 0.20 g of crospovidone, 0.03 g of Cabosil, 0.06 g of corn starch and 0.04 g of magnesium stearate. 2.50 grams of the blend was mixed with 1.0 grams of potassium carbonate. The resulting mixture was compressed into tablets using SC-2 single station tablet press from Key International; each tablet had hardness of 8-12 Kp. Each tablet weighed 350 mg and contained 100 mg of HCl salt of OC-1.

### Example 7

8.96 g of HCl salt of OC-1, 0.24 g of AQOAT, MG type, 1.12 g of Tween 80, and 0.52 g of vitamin E TPGS were dissolved into 80 mL of acetone. A separate solution of 1.12 g of Tween 80 and 0.48 g of vitamin E TPGS was dissolved into 10 mL of acetone. The 10 mL solution was first sprayed onto 4.00 g of sodium bicarbonate and 8.00 g of sodium carbonate using a fluidized bed granulation apparatus. Then, the solution containing HCl salt of OC-1 was sprayed and dried to obtain mixture of fine powder and small granules. 1.83 g of mixture was thoroughly blended with 0.12 g of crospovidone, 0.09 g of Avicel PH101, 0.24 g of corn starch, 0.09 g pregelatinized starch, 0.03 g of Cabosil, 0.30 g of sodium carbonate, 0.30 g of sodium bicarbonate, 0.08 g of sodium lauryl sulfate, and 0.02 g of magnesium stearate. The resulting mixture was compressed into tablets using SC-2 single station tablet press from Key International; each tablet had hardness of 8-12 Kp. Each tablet weighed 515 mg and contained 100 mg of HCl salt of OC-1.

### Example 8

8.96 g of HCl salt of OC-1, 0.24 g of AQOAT, MG type, 2.24 g of Tween 80, and 0.88 g of vitamin E TPGS were dissolved into 80 mL of acetone. The solution was sprayed onto 4.00 g of Avicel PH101, 4.00 g of lactose, 1.84 g of corn starch, 0.24 g of Cabosil, and 3.20 g of crospovidone using a fluidized bed granulation apparatus and dried to obtain mixture of fine powder and small granules. 0.96 g of mixture was thoroughly blended with 0.03 g of Avicel PH101, 0.01 g of corn starch, 0.01 g of Cabosil, 0.30 g of sodium carbonate, 0.15 g of sodium bicarbonate, 0.04 g of sodium lauryl sulfate, and 0.01 g of magnesium stearate. The resulting mixture was compressed into tablets using SC-2 single station tablet press from Key International; each tablet had hardness of 8-12 Kp. Each tablet weighed 548 mg and contained 100 mg of HCl salt of OC-1.

### Example 9

4.48 g of HCl salt of OC-1, 0.02 g of HPMCAS, 0.56 g of Tween 80, and 0.56 g of vitamin E TPGS were dissolved into 20 ml of acetone. The solution was sprayed onto 1.58 g of Avicel PH101, 1.58 g of lactose, and 0.56 g of crospovidone using a fluidized bed granulation apparatus and dried to obtain mixture of fine powder and small granules. 2.00 g of mixture was thoroughly blended with 0.28 g of Avicel PH101, 0.28 g of lactose, 0.08 g of crospovidone, and 0.01 g of magnesium stearate. The resulting mixture was compressed into tablets using SC-2 single station tablet press from Key International; each tablet had hardness of 8-12 Kp. Each tablet weighed 250 mg and contained 100 mg of HCl salt of OC-1.

### Example 10

8.96 g of HCl salt of OC-1, 0.12 g of AQOAT, MG type, 1.12 g of Tween 80, and 0.44 g of vitamin E TPGS were dissolved into 80 mL of acetone. A separate solution of 0.12 g of AQOAT, MG type, 1.12 g of Tween 80, and 0.44 g of vitamin E TPGS was dissolved into 10 mL of acetone. The 10 mL solution was first sprayed onto 4.00 g of Avicel PH101, 4.00 g of lactose, and 3.60 g of crospovidone using a fluidized bed granulation apparatus. Then, the solution containing HCl salt of OC-1 was sprayed and dried to obtain mixture of fine powder and small granules. 2.99 g of mixture was thoroughly blended with 0.11 g of Avicel PH101, 0.11 g of pregelatinized starch, 0.60 g of corn starch, 0.20 g of crospovidone, 0.06 g of Cabosil, 1.50 g of sodium carbonate, 0.90 g of sodium bicarbonate, 0.15 g of sodium lauryl sulfate, and 0.03 g of magnesium stearate. The resulting mixture was compressed into tablets using SC-2 single station tablet press from Key International; each tablet had hardness of 8-12 Kp. Each tablet weighed 665 mg and contained 100 mg of HCl salt of OC-1.

### Example 11

8.96 g of HCl salt of OC-1, 0.10 g of AQOAT, MG type, 1.00 g of Tween 80, and 0.48 g of vitamin E TPGS were dissolved into 80 mL of acetone. A separate solution of 1.00 g of Tween 80 and 0.52 g of vitamin E TPGS was dissolved into 10 mL of acetone. The 10 mL solution was first sprayed onto 2.32 g of Avicel PH101, 2.32 g of lactose, 1.20 g of corn starch, 0.20 g of Cabosil, and 2.24 g of crospovidone using a fluidized bed granulation apparatus. Then, the solution containing HCl salt of OC-1 was sprayed and dried to obtain mixture of fine powder and small granules. 1.53 g of mixture was thoroughly blended with 0.16 g of Avicel PH101, 0.16 g of corn starch, 0.16 g of crospovidone, 0.02 g of Cabosil, 0.60 g of sodium carbonate, 0.30 g of sodium bicarbonate, 0.08 g of sodium lauryl sulfate, and 0.02 g of magnesium stearate. The resulting mixture was compressed into tablets using SC-2 single station tablet press from Key International; each tablet had hardness of 8-12 Kp. Each tablet weighed 499 mg and contained 100 mg of HCl salt of OC-1.

### Example 12

6.72 g of HCl salt of OC-1, 0.6 g of AQOAT, MG type, 0.90 g of Vitamin E TPGS, and 0.9 g of Tween 80 were dissolved in 50 mL of acetone. The solution was sprayed onto 9.00 g of sodium carbonate and 9.00 g of sodium bicarbonate using a fluidized bed granulation apparatus and dried to obtain a mixture of fine powder and small granules. 9.02 g of mixture was thoroughly blended with 1.09 g of Avicel PH101, 0.55 g of lactose, 0.76 g of crospovidone, and 0.03 g of magnesium stearate. The resulting mixture was compressed into tablets using SC-2 single station tablet press from Key International; each tablet had hardness of 8-12 Kp. Each tablet weighed 600 mg and contained 100 mg of HCl salt of OC-1.

### Example 13

8.96 g of HCl salt of OC-1, 0.24 g of AQOAT, MG type, 1.12 g of Tween 80, and 0.52 g of vitamin E TPGS were dissolved into 80 mL of acetone. A separate solution of 1.12 g of Tween 80 and 0.48 g of vitamin E TPGS was dissolved into 10 mL of acetone. The 10 mL solution was first sprayed onto 4.00 g of sodium bicarbonate and 8.00 g of sodium carbonate using a fluidized bed granulation apparatus. Then, the solution containing the HCl salt of OC-1 was sprayed and dried to obtain mixture of fine powder and small granules. 1.83 g of mixture was thoroughly blended with 0.12 g of crospovidone, 0.09 g of Avicel PH101, 0.30 g of corn starch, 0.09 g pregelatinized starch, 0.03 g of Cabosil, 0.30 g of sodium carbonate, 0.15 g of sodium bicarbonate, 0.08 g of sodium lauryl sulfate, and 0.02 g of magnesium stearate. The resulting mixture was compressed into tablets using SC-2 single station tablet press from Key International; each tablet had hardness of 8-12 Kp. Each tablet weighed 500 mg and contained 100 mg of HCl salt of OC-1.

### Example 14

1.12 g of HCl salt of OC-1 was dissolved in a mixture of 0.12 g of Plasdone K29-32, 0.45 g of Tween 80, 0.86 g of vitamin E TPGS, and 1.00 g of trisodium phosphate in 20 mL of water. 0.95 g of Cabosil was added to form a suspension and was mixed for 1 minute on a vortex. The water was evaporated in a rotavapor under vacuum to obtain dry powder. The powder was grinded with pestle in a mortal and passed through #20 size mesh screen. The screened powder was thoroughly blended with 0.25 of Avicel PH101 and 0.25 g of croscarmellose sodium. The resulting mixture was filled into size 0 hard gelatin capsules. Each capsule contained 500 mg of powder and 100 mg of HCl salt of OC-1.

### Example 15

Example 15 was identical to Example 14 except for no Avicel PH101 or croscarmellose sodium was added to the screened powder. Each capsule contained 450 mg of powder and 100 mg of HCl salt of OC-1.

### Example 16

8.96 g of HCl salt of OC-1, 0.12 g of AQOAT, MG type, 1.12 g of Tween 80, and 0.44 g of vitamin E TPGS were dissolved into 80 mL of acetone. A separate solution of 0.12 g of AQOAT, MG type, 1.12 g of Tween 80, and 0.44 g of vitamin E TPGS was dissolved into 10 mL of acetone. The 10 mL solution was first sprayed onto 4.00 g of Avicel PH101, 4.00 g of lactose, and 3.60 g of crospovidone using a fluidized bed granulation apparatus. Then, the solution containing HCl salt of OC-1 was sprayed and dried to obtain mixture of fine powder and small granules. 2.99 g of mixture was thoroughly blended with 0.11 g of Avicel PH101, 0.30 g of pregelatinized starch, 0.60 g of corn starch, 0.30 g of crospovidone, 0.06 g of Cabosil, 1.50 g of sodium carbonate, 0.50 g of sodium bicarbonate, 0.15 g of sodium lauryl sulfate, and 0.03 g of magnesium stearate. The resulting mixture was compressed into tablets using SC-2 single station tablet press from Key International; each tablet had hardness of 8-12 Kp. Each tablet weighed 654 mg and contained 100 mg of HCl salt of OC-1.

### Example 17

8.96 g of HCl salt of OC-1, 0.12 g of AQOAT, MG type, 1.12 g of Tween 80, and 0.44 g of vitamin E TPGS were dissolved into 80 mL of acetone. A separate solution of 0.12 g of AQOAT, MG type, 1.12 g of Tween 80, and 0.44 g of vitamin E TPGS was dissolved into 10 mL of acetone. The 10 mL solution was first sprayed onto 4.00 g of Avicel PH101, 4.00 g of lactose, and 3.60 g of crospovidone using a fluidized bed granulation apparatus. Then, the solution containing HCl salt of OC-1 was sprayed and dried to obtain mixture of fine powder and small granules. 2.99 g of mixture was thoroughly blended with 0.11 g of Avicel PH101, 0.11 g of pregelatinized starch, 0.11 g of corn starch, 0.20 g of crospovidone, 0.06 g of Cabosil, 1.00 g of sodium carbonate, 0.50 g of sodium bicarbonate, 0.15 g of sodium lauryl sulfate, and 0.03 g of magnesium stearate. The resulting mixture was compressed into tablets using SC-2 single station tablet press from Key International; each tablet had hardness of 8-12 Kp. Each tablet weighed 545 mg and contained 100 mg of HCl salt of OC-1.

### Example 18

4.48 g of HCl salt of OC-1, 0.17 g of AQOAT, MG type, 1.64 g of Tween 80, and 0.60 g of Vitamin E TPGS were dissolved into 40 mL of acetone. The solution was sprayed onto 4.00 g of sodium bicarbonate, 6.00 g of sodium carbonate, 2.80 g of crospovidone, and 4.48 g of Avicel PH101 using a fluidized bed granulation apparatus and dried to obtain mixture of fine powder and small granules. 6.04 g of mixture was thoroughly blended with 0.40 g of crospovidone, 0.45 g of Avicel PH101, 0.30 g of corn starch, 0.26 g pregelatinized starch, 0.10 g of Cabosil, 0.50 g of sodium carbonate, 0.5 g of sodium bicarbonate, 0.21 g of sodium lauryl sulfate, and 0.04 g of magnesium stearate. The resulting mixture was compressed into tablets using SC-2 single station tablet press from Key International; each tablet had hardness of 8-12 Kp. Each tablet weighed 880 mg and contained 100 mg of HCl salt of OC-1.

### Example 19

4.48 g of OC-1, 0.17 g of AQOAT, MG type, 1.64 g of Tween 80, and 0.60 g of vitamin E TPGS were dissolved into 40 mL of acetone. The solution was sprayed onto 4.00 g of sodium bicarbonate, 2.80 g crospovidone, and 6.00 g of sodium carbonate using a fluidized bed granulation apparatus and dried to obtain mixture of fine powder and small granules. 3.62 g of mixture was thoroughly blended with 0.24 g of crospovidone, 0.27 g of Avicel PH101, 0.16 g pregelatinized starch, 0.30 g of sodium carbonate, 0.30 g of sodium bicarbonate, 0.36 g of corn starch, 0.12 g of Cabosil, 0.12 g of sodium lauryl sulfate, and 0.03 g of magnesium stearate. The resulting mixture was compressed into tablets using SC-2 single station tablet press from Key International; each tablet had hardness of 8-12 Kp. Each tablet weighed 704 mg and contained 80 mg of HCl salt of OC-1.

### Example 20

5.6 g of HCl salt of OC-1, 5.0 g of AQOAT, LG type, and 0.5 g of Tween 80 were dissolved in 50 mL of acetone. The solution was sprayed dried in a spray dryer (Niro SDMicro spray drier, glass drying chamber, and filter housing; single pass nitrogen gass, 0.5 mm liquid insert, single point collection, at 1.0 bar; Inlet temperature between 70 and 80° C. for acetone) and dried to obtain fine powder. 6.66 g of the powder was thoroughly blended with 0.97 g of crospovidone, 0.97 g of Avicel PH101, 4.50 g of sodium carbonate, and 3.0 g of sodium bicarbonate. The powder was compressed in a tablet press, milled, and passed through #40 mesh screen. The powder was then blended with 0.91 g of crospovidone, 0.91 g of Avicel PH101, 0.91 g of pregelatinized starch, 1.50 g of sodium carbonate, 1.50 g of sodium bicarbonate, 0.11 g of Cabosil, and 0.11 g of magnesium stearate. The resulting mixture was compressed into tablets using SC-2 single station tablet press from Key International; each tablet had hardness of 8-12 Kp. Each tablet weighed 588 mg and contained 80 mg of HCl salt of OC-1.

### Example 21

5.6 g of HCl salt of OC-1, 5.0 g of AQOAT, MG type, and 0.5 g of Tween 80 were dissolved in 50 mL of acetone. The solution was sprayed dried in a spray dryer (see conditions in Example 20) and dried to obtain fine powder. 1.11 g of the powder was thoroughly blended with 0.16 g of crospovidone, 0.16 g of microcrystalline cellulose, 0.49 g of sodium carbonate, and 0.49 g of sodium bicarbonate. The powder was compressed in a tablet press, milled and passed through #40 mesh screen. The powder was then blended with 0.09 g of crospovidone, 0.14 g of Avicel PH101, 0.05 g of pregelatinized starch, 0.26 g of sodium carbonate, 0.26 g of sodium bicarbonate, 0.02 g of Cabosil, and 0.02 g of magnesium stearate. The resulting mixture was compressed into tablets using SC-2 single station tablet press from Key International; each tablet had hardness of 8-12 Kp. Each tablet weighed 650 mg and contained 100 mg of HCl salt of OC-1.

### Example 22

5.6 g of HCl salt of OC-1, 5.0 g of AQOAT, MG type, and 0.5 g of Tween 80 were dissolved in 50 mL of acetone. The solution was sprayed dried in a spray dryer (see conditions in Example 20) and dried to obtain fine powder. 1.11 g of the powder was thoroughly blended with 0.16 g of crospovidone, 0.16 g of Avicel PH101, 0.75 g of sodium carbonate, and 0.50 g of sodium bicarbonate. The powder was compressed in a tablet press, milled, and passed through #40 mesh screen. The powder was then blended with 0.15 g of crospovidone, 0.15 g of Avicel PH101, 0.15 g of pregelatinized starch, 0.25 g of sodium carbonate, 0.25 g of sodium bicarbonate, 0.02 g of Cabosil, and 0.02 g of magnesium stearate. The resulting mixture was compressed into tablets using SC-2 single station tablet press from Key International; each tablet had hardness of 8-12 Kp. Each tablet weighed 588 mg and contained 80 mg of HCl salt of OC-1.

### Example 23

5.6 g of HCl salt of OC-1, 5.0 g of AQOAT, MG type, and 0.5 g of Tween 80 were dissolved in 50 mL of acetone. The solution was sprayed dried in a spray dryer (see conditions in Example 20) and dried to obtain fine powder. 1.33 g of the powder was thoroughly blended with 0.36 g of crospovidone, 0.26 g of Avicel PH101, 0.30 g of sodium carbonate, 0.60 g of sodium bicarbonate, 0.30 g of corn starch, 0.30 g pregelatinized starch, and 0.12 g of sodium lauryl sulfate. The powder was compressed in a tablet press, milled, and passed through #40 mesh screen. The powder was then blended with 0.30 g of crospovidone, 0.26 g of Avicel PH101, 0.27 g of pregelatinized starch, 0.30 g of corn starch, 0.30 g of sodium carbonate, 0.30 g of sodium bicarbonate, 0.06 g of Cabosil, and 0.03 g of magnesium stearate. The resulting mixture was compressed into tablets using SC-2 single station tablet press from Key International; each tablet had hardness of 8-12 Kp. Each tablet weighed 800 mg and contained 80 mg of HCl salt of OC-1.

### Example 24

11.2 g of HCl salt of OC-1 was suspended in a solution of 1.0 g of Pluronic F127, 1.0 g of Tween 80, and 0.5 g of Plasdone K29-32 in 100 mL of water. The mixture was milled in a mill (DYNO-MILL Multilab) to produce nanosuspension. The nanosuspension was filtered through a 1.2 micron syringe filter. The nanosuspension was assayed to contain 75 mg/mL HCl salt of OC-1. The solution was sprayed dried in a spray dryer (see conditions in Example 20 with the exception that the inlet temperature was 120° C.) and dried to obtain fine powder. 1.09 g of the powder was thoroughly blended with 0.37 g of crospovidone, 0.55 g of Avicel PH101, 0.05 g of Cabosil, and 0.01 g of magnesium stearate. The resulting mixture was compressed on top of pre-pressed tablet (hardness 2-4 Kp) containing 0.20 g of sodium carbonate, 0.10 g of sodium bicarbonate, 0.03 g of crospovidone, and 0.01 g of magnesium stearate, forming double layer tablets using SC-2 single station tablet press from Key International; each tablet had hardness of 8-12 Kp. Each tablet weighed 600 mg and contained 100 mg of HCl salt of OC-1.

### Example 25

5.6 g of HCl salt of OC-1, 15.0 g of AQOAT, MG type, and 0.5 g of Tween 80 were dissolved in 50 mL of acetone. The solution was sprayed dried in a spray dryer (see conditions in Example 20) and dried to obtain fine powder. 8.24 g of the powder was thoroughly blended with 0.48 g of crospovidone, 0.32 g of pregelatinized starch, 0.12 g of Cabosil, 2.00 g of potassium carbonate, and 0.06 g of magnesium stearate. The resulting mixture was filled into size 0 hard gelatin capsules. Each capsule had 560 mg of material and contained 100 mg of HCl salt of OC-1.

### Example 26

5.6 g of OC-1, 5.0 g of AQOAT, MG type, and 0.5 g of Tween 80 were dissolved in 50 mL of acetone. The solution was sprayed dried in a spray dryer (see conditions in Example 20) and dried to obtain fine powder. 4.24 g of the powder was thoroughly blended with 0.30 g of crospovidone, 0.20 g of pregelatinized starch, 0.07 g of Cabosil, 2.00 g of potassium carbonate, and 0.04 g of magnesium stearate and filled into size 0 hard gelatin capsules. Each capsule had 342 mg of material and contained 100 mg of OC-1.

The products of comparative Examples A through K, and Examples 1 through 26 were analyzed for in vivo bioavailability using dogs (male, beagle dogs (n=3) weighing 6.5-9.0 kg). The dose was administered orally to animals in the fasted state (where food was withheld overnight). Following dosing, blood samples (1.0 mL) for pharmacokinetic evaluation were collected via venipuncture from each animal at predose (O), and at 0.5, 1, 2, 3, 4, 6, 8, 12, and 24 hours into lithium-hepranized tubes. After each time point, all blood samples were collected, processed, and frozen at about −70° C.

The concentrations of the compound in dog plasma were determined by a LC-MS/MS assay following a protein precipitation step with acetonitrile. Pharmacokinetic analysis was performed using the WinNonlin™ software program (Pharsight, Inc. Mountain View, Calif.). The area under the plasma concentration-time curve (AUC₀₋ₜ) is calculated from the first time point (0 min) up to the last time point with measurable drug concentration. The AUC_{0-inf} was calculated as the sum of AUC₀₋ₜ and Cpred/λz, where Cpred was the predicted concentration at the time of the last quantifiable concentration.

The results of analysis of Examples A through K are shown in Table 1. The results of analysis of Examples 1 through 19 (sprayed onto fluidized bed or rotovap) are shown in Table 2, and the results of analysis of Examples 20 through 26 (spray dried) are shown in Table 3.

Example K presented herein is a solution formulation that represents the idealized or targeted pharmacokinetic profile for the solid compositions of the invention. As seen in Table 1, the Cmax achieved with Example K is 1330 ng/mL, and the AUC₀₋ₜ and AUC_{0-inf} are 5043 hr*ng/mL and 5149 hr*ng/mL respectively.

Examples A-J, on the other hand, represent solid compositions not within the scope of the invention. For example, none of the compositions comprise at least one pharmaceutically acceptable basic excipient, among other differences. As seen from Table 1, the Cmax for Examples A-J range from 9-221 ng/mL, and the AUC₀₋ₜ and AUC_{0-inf} range from 42-1378 hr*ng/mL and 121-1598 hr*ng/mL respectively.

Examples 1-19 (Table 2) are compositions that utilize spraying onto fluidized beds or rotovaps and show results that are improved over the comparable compositions of Examples A-J. For example, Example B and Example 2 are similar in composition and method of preparation but for the addition of potassium carbonate, a basic excipient, to the composition of Example 2. As seen in Tables 1 and 2, the Cmax for Example 2 is more than four times greater than that of Example B. Similarly, the AUC₀₋ₜ and AUC_{0-inf} for Example 2 are each almost four times greater than those of Example B.

Examples 20-26 (Table 3) are compositions that utilize spray drying and show results that are improved over the comparable compositions of Examples A-J. For example, Example J and Example 21 are similar in composition and method of preparation but the composition of Example 21 further comprises sodium carbonate and sodium bicarbonate. As seen in Tables 1 and 3, the Cmax for Example 21 is more than three times greater than that of Example J. Similarly, the AUC₀₋ₜ and AUC_{0-inf} for Example 21 are each more than double those of Example J.

TABLE 1

| | Form/Amt. | | | | | | | |
|---|---|---|---|---|---|---|---|---|
| | of HCl | | | | | | | |
| | salt of | | Solvent/ | | | | | |
| | Formula | Binding | Evaporation | Basic | Dose | Cₘₐₓ | AUC₀₋ₜ | AUC_{0-inf} |
| Ex. | (I) | Agent | Method | Excipient | (mg/kg) | (ng/mL) | (hr * ng/mL) | (hr * ng/mL) |
| A | Capsules/ | none | none | none | 5.4 | 27 | 125 | 137 |
| | 100 mg | | | | | | | |
| B | Tablets/ | PVP | Ethanol/sprayed | none | 12.7 | 9 | 42 | 131 |
| | 100 mg | | onto fluidized | | | | | |
| | | | bed | | | | | |
| C | Tablets/ | PVP | Ethanol/sprayed | none | 11.6 | 28 | 225 | 121 |
| | 90 mg | | onto fluidized | | | | | |
| | | | bed | | | | | |
| D | Tablets/ | PVP | Ethanol/sprayed | none | 13.0 | 49 | 407 | 513 |
| | 100 mg | | onto fluidized | | | | | |
| | | | bed | | | | | |
| E | Tablets/ | PVP | Water | none | 11.8 | 59 | 401 | 334 |
| | 90 mg | | (nanosuspension)/ | | | | | |
| | | | sprayed onto | | | | | |
| | | | fluidized bed | | | | | |
| F | Capsules/ | PVP | Ethanol/rotovap | none | 4.8 | 75 | 380 | 407 |
| | 40 mg | | | | | | | |
| G | Tablets/ | PVP | Water | none | 11.7 | 144 | 534 | 597 |
| | 90 mg | | (nanosuspension)/ | | | | | |
| | | | sprayed onto | | | | | |
| | | | fluidized bed | | | | | |
| H | Capsules/ | HPMC | Wet granulation | none | 10.9 | 54 | 339 | 362 |
| | 100 mg | (E3) | | | | | | |
| I | Capsules/ | PVP | Ethanol/rotovap | none | 4.8 | 75 | 380 | 407 |
| | 40 mg | | | | | | | |
| J | Tablets/ | HPMCAS | Acetone/spray | none | 11.3 | 221 | 1378 | 1598 |
| | 100 mg | (1:1 | dried | | | | | |
| | | MG) | | | | | | |
| K | Solution/ | PVP | Water | none | 9.0 | 1330 | 5043 | 5149 |
| | 100 mg | | (nanosuspension)/ | | | | | |
| | | | none | | | | | |

TABLE 2

| | Form/Amt. | | | | | | | |
|---|---|---|---|---|---|---|---|---|
| | of HCl | | | | | | | |
| | salt of | | Solvent/ | | | | | |
| | Formula | Binding | Evaporation | Basic | Dose | Cₘₐₓ | AUC₀₋ₜ | AUC_{0-inf} |
| Ex. | (I) | Agent | Method | Excipient | (mg/kg) | (ng/mL) | (hr * ng/mL) | (hr * ng/mL) |
| 1 | Capsules/ | PVP | Water | Potassium | 13.0 | 214 | 970 | 1028 |
| | 100 mg | | (nanosuspension)/ | Carbonate | | | | |
| | | | sprayed | | | | | |
| | | | onto | | | | | |
| | | | fluidized | | | | | |
| | | | bed | | | | | |
| 2 | Tablets/ | PVP | Ethanol/ | Potassium | 13.3 | 231 | 1582 | 1924 |
| | 100 mg | | sprayed | Carbonate | | | | |
| | | | onto | | | | | |
| | | | fluidized | | | | | |
| | | | bed | | | | | |
| 3 | Tablets/ | PVP | Water | Potassium | 13.0 | 240 | 1759 | 2055 |
| | 100 mg | | (nanosuspension)/ | Carbonate | | | | |
| | | | sprayed | | | | | |
| | | | onto | | | | | |
| | | | fluidized | | | | | |
| | | | bed | | | | | |
| 4 | Capsules/ | PVP | Ethanol/ | Potassium | 11.7 | 263 | 1456 | 1532 |
| | 100 mg | | sprayed | Carbonate | | | | |
| | | | onto | | | | | |
| | | | fluidized | | | | | |
| | | | bed | | | | | |
| 5 | Tablets/ | HPMC | Acetone/ | Potassium | 12.1 | 278 | 1613 | 1778 |
| | 100 mg | E3 | sprayed | Carbonate | | | | |
| | | | onto | | | | | |
| | | | fluidized | | | | | |
| | | | bed | | | | | |
| 6 | Tablets/ | PVP | Ethanol/ | Potassium | 12.7 | 366 | 1790 | 1981 |
| | 100 mg | | sprayed | Carbonate | | | | |
| | | | onto | | | | | |
| | | | fluidized | | | | | |
| | | | bed | | | | | |
| 7 | Tablets/ | HPMCAS | Acetone/ | Sodium | 11.7 | 392 | 1603 | 1655 |
| | 100 mg | (0.5%) | sprayed | Carbonate, | | | | |
| | | MG | onto | Sodium | | | | |
| | | | fluidized | bicarbonate | | | | |
| | | | bed | | | | | |
| 8 | Tablets/ | HPMCAS | Acetone/ | Sodium | 11.0 | 462 | 2464 | 2527 |
| | 100 mg | (0.5%) | sprayed | Carbonate, | | | | |
| | | MG | onto | Sodium | | | | |
| | | | fluidized | Bicarbonate | | | | |
| | | | bed | | | | | |
| 9 | Tablets/ | HPMCAS | Acetone/ | Potassium | 12.1 | 520 | 1961 | 2001 |
| | 100 mg | (0.25%) | sprayed | Carbonate | | | | |
| | | | onto | | | | | |
| | | | fluidized | | | | | |
| | | | bed | | | | | |
| 10 | Tablets/ | HPMCAS | Acetone/ | Sodium | 11.7 | 540 | 2613 | 2650 |
| | 100 mg | (0.5%) | sprayed | Carbonate, | | | | |
| | | MG | onto | Sodium | | | | |
| | | | fluidized | Bicarbonate | | | | |
| | | | bed | | | | | |
| 11 | Tablets/ | HPMCAS | Acetone/ | Sodium | 12.2 | 547 | 1811 | 1831 |
| | 100 mg | (0.25%) | sprayed | Carbonate, | | | | |
| | | MG | onto | Sodium | | | | |
| | | | fluidized | Bicarbonate | | | | |
| | | | bed | | | | | |
| 12 | Tablets/ | HPMCAS | Acetone/ | Sodium | 11.4 | 576 | 2162 | 2479 |
| | 100 mg | | sprayed | Carbonate, | | | | |
| | | | onto | Sodium | | | | |
| | | | fluidized | Bicarbonate | | | | |
| | | | bed | | | | | |
| 13 | Tablets/ | HPMCAS | Acetone/ | Sodium | 12.6 | 718 | 3548 | 3572 |
| | 100 mg | (0.5%) | sprayed | Carbonate, | | | | |
| | | MG | onto | Sodium | | | | |
| | | | fluidized | Bicarbonate | | | | |
| | | | bed | | | | | |
| 14 | Capsules/ | PVP | Water/ | Trisodium | 10.6 | 827 | 2488 | 2571 |
| | 100 mg | | rotovap | phosphate | | | | |
| 15 | Capsules/ | PVP | Water/ | Trisodium | 8.4 | 746 | 2760 | 2777 |
| | 100 mg | | rotovap | phosphate | | | | |
| 16 | Tablets/ | HPMCAS | Acetone/ | Sodium | 11.8 | 865 | 3544 | 3570 |
| | 100 mg | (0.5%) | sprayed | Carbonate, | | | | |
| | | MG | onto | Sodium | | | | |
| | | | fluidized | Bicarbonate | | | | |
| | | | bed | | | | | |
| 17 | Tablets/ | HPMCAS | Acetone/ | Sodium | 11.5 | 1343 | 3996 | 4011 |
| | 100 mg | (0.5%) | sprayed | Carbonate, | | | | |
| | | MG | onto | Sodium | | | | |
| | | | fluidized | Bicarbonate | | | | |
| | | | bed | | | | | |
| 18 | Tablets/ | HPMCAS | Acetone/ | Sodium | 9.4 | 1344 | 6021 | 6141 |
| | 80 mg | (0.5%) | sprayed | Carbonate, | | | | |
| | | MG | onto | Sodium | | | | |
| | | | fluidized | Bicarbonate | | | | |
| | | | bed | | | | | |
| 19 | Tablets/ | HPMCAS | Acetone/ | Sodium | 10.3 | 1409 | 6333 | 6371 |
| | 80 mg | (0.5%) | sprayed | Carbonate, | | | | |
| | | MG | onto | Sodium | | | | |
| | | | fluidized | Bicarbonate | | | | |
| | | | bed | | | | | |

TABLE 3

| | Form/Amt. | | | | | | | |
|---|---|---|---|---|---|---|---|---|
| | of HCl | | | | | | | |
| | salt of | | Solvent/ | | | | | |
| | Formula | Binding | Evaporation | Basic | Dose | Cₘₐₓ | AUC₀₋ₜ | AUC_{0-inf} |
| Ex. | (I) | Agent | Method | Excipient | (mg/kg) | (ng/mL) | (hr * ng/mL) | (hr * ng/mL) |
| 20 | Tablets/ | HPMCAS | Acetone/ | Sodium | 9.9 | 223 | 1149 | 1288 |
| | 80 mg | (1:1) | spray dried | Carbonate, | | | | |
| | | LG | | Sodium | | | | |
| | | | | Bicarbonate | | | | |
| 21 | Tablets/ | HPMCAS | Acetone/ | Sodium | 11.3 | 685 | 3253 | 3362 |
| | 100 mg | (1:1) | spray dried | Carbonate, | | | | |
| | | MG | | Sodium | | | | |
| | | | | Bicarbonate | | | | |
| 22 | Tablets/ | HPMCAS | Acetone/ | Sodium | 9.3 | 728 | 2836 | 2919 |
| | 80 mg | (1:1) | spray dried | Carbonate, | | | | |
| | | MG | | Sodium | | | | |
| | | | | Bicarbonate | | | | |
| 23 | Tablets/ | HPMCAS | Acetone/ | Sodium | 9.2 | 864 | 3590 | 3618 |
| | 80 mg | (1:1) | spray dried | Carbonate, | | | | |
| | | MG | | Sodium | | | | |
| | | | | Bicarbonate | | | | |
| 24 | Tablets/ | PVP | Water | Sodium | 11.6 | 1037 | 3715 | 3809 |
| | 100 mg | | (nanosuspension)/ | Carbonate, | | | | |
| | | | spray dried | Sodium | | | | |
| | | | | Bicarbonate | | | | |
| 25 | Capsules/ | HPMCAS | Acetone/ | Potassium | 12.9 | 1081 | 3911 | 4013 |
| | 100 mg | (1:3) | spray dried | Carbonate | | | | |
| 26 | Capsules/ | HPMCAS | Acetone/ | Potassium | 11.9 | 1172 | 4795 | 4854 |
| | 100 mg | (1:1) | spray dried | Carbonate | | | | |

1. A solid composition comprising (S)-3-(4′-Cyano-biphenyl-4-yl)-2-{[(3S,7S)-3-[4-(3,4-dichloro-benzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid or a salt thereof and at least one pharmaceutically acceptable basic excipient.

2. The solid composition of claim 1, wherein the at least one pharmaceutically acceptable basic excipient is selected from trisodium phosphate, potassium carbonate, sodium carbonate, and sodium bicarbonate.

3. The solid composition of claim 1, further comprising at least one water-soluble surfactant.

4. The solid composition of claim 3, wherein the at least one water-soluble surfactant is selected from polyoxyethylene sorbitan fatty acid esters, polyoxyethylene derivatives of natural oils and waxes, polyethylene glycol fatty acid esters, propylene glycol fatty acid mono- or diesters, sorbitan fatty acid esters, polyoxyethylene-polyoxypropylene copolymer and block copolymer surfactants, sulfuric acid alkyl ester salts, and bile acid salts.

5. The solid composition of claim 1, further comprising a pharmaceutically acceptable carrier.

6. The solid composition of claim 1, wherein the composition is in the form of powder.

7. The solid composition of claim 1, wherein the composition is in the form of a capsule or tablet.

8. The solid composition of claim 1, wherein the (S)-3-(4′-Cyano-biphenyl-4-yl)-2-{[(3S,7S)-3-[4-(3,4-dichloro-benzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid or the salt thereof is in its amorphous form.

9. A solid composition comprising an evaporation residue of (S)-3-(4′-Cyano-biphenyl-4-yl)-2-{[(3S,7S)-3-[4-(3,4-dichloro-benzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid or a salt thereof and at least one pharmaceutically acceptable basic excipient.

10. The solid composition of claim 9, wherein the at least one pharmaceutically acceptable basic excipient is selected from trisodium phosphate, potassium carbonate, sodium carbonate, and sodium bicarbonate.

11. The solid composition of claim 9, wherein the evaporation residue further comprises at least one pharmaceutically acceptable polymeric stabilizing agent.

12. The solid composition of claim 11, wherein the at least one pharmaceutically acceptable polymeric stabilizing agent is selected from polyvinylpyrrolidone (PVP), hydroxypropylmethyl cellulose acetate succinate (HPMCAS), hydroxypropylmethyl cellulose phthalate (HPMCP), hydroxypropylmethyl cellulose (HPMC), poloxamers, hydroxypropyl methyl cellulose acetate, hydroxypropyl methyl cellulose, hydroxypropyl cellulose, and hydroxyethyl cellulose acetate, polyacrylates, methyl acrylatemethacrylic acid copolymers, ethyl acrylatemethacrylic acid copolymers, cellulose acetate phthalate, cellulose acetate trimellitate, and carboxymethyl ethyl cellulose.

13. The solid composition of claim 9, further comprising at least one water-soluble surfactant.

14. The solid composition of claim 13, wherein the at least one water-soluble surfactant is selected from polyoxyethylene sorbitan fatty acid esters, polyoxyethylene derivatives of natural oils and waxes, polyethylene glycol fatty acid esters, propylene glycol fatty acid mono- or diesters, sorbitan fatty acid esters, polyoxyethylene-polyoxypropylene copolymer and block copolymer surfactants, sulfuric acid alkyl ester salts, and bile acid salts.

15. The solid composition of claim 9, further comprising a solid pharmaceutically acceptable carrier.

16. The solid composition of claim 9, wherein the composition is in the form of a capsule or tablet.

17. A method of making a solid composition comprising:
mixing (S)-3-(4′-Cyano-biphenyl-4-yl)-2-{[(3S,7S)-3-[4-(3,4-dichloro-benzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid or a salt thereof and at least one pharmaceutically acceptable basic excipient in at least one solvent to form a solution or suspension; and
removing the solvent from the solution or suspension to form a powder.

18. The method of claim 17, wherein during the mixing step at least one pharmaceutically acceptable polymeric stabilizing agent, at least one water-soluble surfactant, or at least one pharmaceutically acceptable ingredient is mixed with (S)-3-(4′-Cyano-biphenyl-4-yl)-2-{[(3S,7S)-3-[4-(3,4-dichloro-benzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid or salt thereof, the at least one basic excipient and the at least one solvent.

19. The method of claim 17, wherein the step of removing the solvent comprises spray drying the solution or suspension to form a powder.

20. The method of claim 19, wherein the spray drying step sprays the solution or suspension onto a solid pharmaceutically acceptable carrier to form a powdered mixture.

21. The method of claim 19, wherein the spray drying step is performed in a spray dryer or a fluid bed dryer/granulator.

22. The method of claim 20, wherein the solid pharmaceutically acceptable carrier comprises a pharmaceutically acceptable basic excipient, a pharmaceutically acceptable inert carrier, or mixtures thereof.

23. The method of claim 20, wherein the powdered mixture further comprises at least one additional pharmaceutical ingredient.

24. The method of claim 17, further comprising the step of tabletizing the powdered mixture.

25. The method of claim 24, wherein tabletizing the powdered mixture forms a multilayer tablet.

26. A method for the treatment of type 2 diabetes or high blood glucose levels, the method comprising administering to a subject a solid composition of claim 1 wherein the solid composition comprises a therapeutically effective amount of (S)-3-(4′-Cyano-biphenyl-4-yl)-2-{[(3S,7S)-3-[4-(3,4-dichloro-benzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid or a salt thereof.

27. A method of lowering blood glucose concentration in a subject comprising administering to a subject a solid composition of claim 1, wherein the solid composition comprises a therapeutically effective amount of (S)-3-(4′-Cyano-biphenyl-4-yl)-2-{[(3S,7S)-3-[4-(3,4-dichloro-benzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid or a salt thereof.

28. A method of stimulating insulin secretion in a subject comprising administering to a subject a solid composition of claim 1, wherein the solid composition comprises a therapeutically effective amount of (S)-3-(4′-Cyano-biphenyl-4-yl)-2-{[(3S,7S)-3-[4-(3,4-dichloro-benzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid or a salt thereof.

29. A monohydrochloride salt of (S)-3-(4′-Cyano-biphenyl-4-yl)-2-{[(3S,7S)-3-[4-(3,4-dichloro-benzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid.

30. A pharmaceutical composition comprising a monohydrochloride salt of (S)-3-(4′-Cyano-biphenyl-4-yl)-2-{[(3S,7S)-3-[4-(3,4-dichloro-benzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid and a at least one pharmaceutically acceptable basic excipient.

31. A method of treating type 2 diabetes comprising administering to a human a monohydrochloride salt of (S)-3-(4′-Cyano-biphenyl-4-yl)-2-{[(3S,7S)-3-[4-(3,4-dichloro-benzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid.

32. A method of lowering blood glucose in a human comprising administering to a human a monohydrochloride salt of (S)-3-(4′-Cyano-biphenyl-4-yl)-2-{[(3S,7S)-3-[4-(3,4-dichloro-benzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid.

33. A method of stimulating insulin secretion in a human comprising administering to a human a monohydrochloride salt of (S)-3-(4′-Cyano-biphenyl-4-yl)-2-{[(3S,7S)-3-[4-(3,4-dichloro-benzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid.
